(19) United States
(12) Patent Application Publication
(10) Pub. No.: US 2026/0309708 A1
(43) Pub. Date: Oct. 8, 2026

(54) COMPOSITIONS AND METHODS FOR DETERMINING THE EFFECTS OF T CELL ENGAGER

(71) Applicant: ARBELE Limited, Shatin, N.T., (HK)

(72) Inventors: John Moonching LUK, Bellevue, WA (US); Chu Qiao CHOW, Hong Kong (HK); Nga Ting LIN; Dennis A. WONG, Hong Kong (HN)

(73) Assignee: ARBELE Limited, Shatin, N.T., (HK)

(21) Appl. No.: 19/476,171

(22) PCT Filed: Apr. 16, 2024
(86) PCT No.: PCT/US2024/024813

§ 371 Date: Oct. 17, 2025

Related U.S. Application Data
(60) Provisional application No. 63/459,937, filed on Apr. 17, 2023.

(51) Int. Cl. G01N 33/569; C12N 5/0783; G01N 33/50; G01N 33/575

(52) U.S. Cl. CPC G01N 33/56972; C12N 5/0636; G01N 33/5011; G01N 33/505; G01N 33/5758; G01N 2333/7051; G01N 2500/10

(57) ABSTRACT

An assaying system to measure T cell engagement and disengagement to target cancer cells by T cell engager (TCE) antibodies, and its subsequent effects on T cells activation and proliferation. The assay platform may be used to screen TCE candidates that form detachable immune synapse and may provide long-term protection.

### CROSS-REFERENCE TO RELATED APPLICATIONS

This application claims the benefit of the filing date of U.S. Provisional Application Ser. No. 63/459,937 filed Apr. 17, 2023, under 35 U.S.C. 119(e), the entire disclosures of which are incorporated by reference herein.

### TECHNICAL FIELD

The present application relates to methods useful for the detection of cancer. In particular, the present application relates to T cell engager (TCE).

### BACKGROUND

The cytotoxic nature of T cell makes it valuable in cancer therapy. A variety of strategies are devised to make use of T cells to treat cancers, such as T cell engager (TCE), chimeric antigen receptor (CAR) T cells, TCR-T cells and more. TCE especially, have been demonstrated to be highly potent: cytotoxicity could be induced at picomolar doses and robust T cell activation could be observed. The first anti-CD3/CD19 TCE, Blinatumomab (Amgen, BLINCYTO®) was approved in 2014 for treating acute lymphoblastic leukemia. Multiple studies have proven TCE induce T cell activation by showing upregulation of CD25 and CD69, and secretion of cytokines like IL-2, TNF-α and IFN-γ. More recently, a novel gp100 peptide-HLA TCR/anti-CD3 fusion protein Tebentafusp (Immunocore, KIMMTRAK®) was approved in 2022 for treatment of metastatic uveal melanoma. The approval of TCE demonstrates the demand for more effective immunotherapeutics. However, despite being a powerful cytotoxic agent, little is known about the long-term effects of TCE stimulation on T cells and there is no evidence of long-term protection against cancer recurrence.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

The application provides, among others, assaying systems, apparatus, methods, and processes for evaluating the efficacy or safety of a T cell engager (TCE). The application further provides biocomplex and compositions.

In one aspect, the application provides assaying systems for evaluating the efficacy and safety of a T cell engager (TCE). In one embodiment, the assaying system comprises a cell culture plate having at least one chamber for seeding a plurality of cells expressing at least a cancer marker in a cell culture medium. The density may be of at least 30,000, 40,000, 50,000, or 100,000 cells per cm² per chamber. The cells are attached to the surface of the cell culture plate.

The cell culture medium comprises a plurality of T-cells and a TCE. In one embodiment, the number of T cells may not exceed two thirds (⅔) of the number of cancer cells seeded. The TCE may be at a concentration of at least 1 ng per ml of the cell culture medium. The TCE may have a first binding specificity to the cancer marker and a second binding specificity to the T-cells.

In one embodiment, the rate of the T-cells attaching to, or detaching from, the cells expressing at least a cancer marker may be measured.

In one embodiment, the assaying system comprises a first cell culture plate having at least one chamber for seeding a plurality of cells expressing at least a cancer marker in a first cell culture medium. The density may be at least 30,000, 50,000, or 100,000 cells per cm² per chamber. The cells may be attached to the surface of the first cell culture plate.

In one embodiment, the first cell culture medium comprises a plurality of T-cells and a TCE. The number of T cells may be about 4 times, 5 times, or 6 times the number of cancer cells seeded. The TCE may be at a concentration of at least 1 ng per ml of the first cell culture medium. The TCE may have a first binding specificity to the cancer marker and a second binding specificity to the T-cells. The cells expressing at least a cancer marker, the T-cells, and the TCE are co-cultured for a co-culturing period to provide activated T-cells.

In one embodiment, the TCE may be configured to bind the T-cells and the cells expressing at least a cancer marker to provide a biocomplex, therefore activating T-cells, killing the cells expressing at least a cancer marker, and causing the cells expressing at least a cancer marker to detach from the first cell culture plate. In one embodiment, the biocomplex including T-cells may be initially attached to the first cell culture plate due to the attachment of the cells expressing at least a cancer marker. Then, by killing the cells expressing at least a cancer marker, the activated T-cells are detached from the first culture plate and released into the first cell culture medium. Therefore, in one embodiment, the first cell culture medium may include activated T-cells detached from the first cell culture plate by killing the cells expressing at least a cancer marker.

The assaying system further comprises a second cell culture plate for measuring the growth rate of the activated T-cells. In one embodiment, the second cell culture plate comprises a second cell culture medium. In one embodiment, the activated T-cells are removed from the first cell culture medium and cultured in the second cell culture medium for an expansion culturing period to provide expanded activated T-cells. In one embodiment, the growth rate is measured by pre-labeling the T-cells with a fluorescent dye and counting the number of expanded activated T-cells by measuring, for example, fluorescence.

The assaying system may further comprise a third cell culture plate for measuring the cytotoxicity of the activated T-cells. In one embodiment, the third cell culture plate comprises the cells expressing at least a cancer marker in a third cell culture medium. In one embodiment, the activated T-cells are removed from the first or the second cell culture medium and co-grown in the third cell medium with the cells expressing at least a cancer marker for a co-growing period. In one embodiment, the cytotoxicity of the activated T-cells is measured by counting the cell death of the cells expressing at least a cancer marker.

In one embodiment, the co-culturing period is from about half a day to about 2 days.

In one embodiment, the expansion culturing period is from about 2 days to about 7 days.

In one embodiment, the co-growing period is from about half a day to about 1 day.

In one embodiment, the cells expressing a cancer marker comprise cancer cells. In one embodiment, the cancer marker comprises CDH17. In one embodiment, the cancer cells comprise gastrointestinal cancer cells, gastric cancer cells, colorectal cancer cells, esophageal cancer cells, pancreatic cancer cells, hepatobilliary cancer cells, small cell lung cancer cells, ovarian cancer cells, bladder cancer cells, neuroendocrine tumor cells, adenoma cancer cells, or a combination thereof.

In one embodiment, the T-cells comprise T lymphocytes derived from peripheral blood mononuclear cells, cytotoxic T cells, helper T cells, natural killer T cells, or a combination thereof. In one embodiment, the T-cells express CD3.

In one embodiment, the TCE comprises a bi-specific antibody or a multi-specific antibody. In one embodiment, the bispecific antibody has a binding specificity to CD3 and CDH17.

In another aspect, the application provides biocomplexes. In one embodiment, the biocomplex comprises essentially an immune synapse. In one embodiment, the biocomplex comprises the TCE bound to the T-cell and the cell expressing at least a cancer marker. In one embodiment, the biocomplex comprises the TCE bound to the T-cell. In one embodiment, the biocomplex comprises TCE bound to the cells expressing at least a cancer marker.

In one embodiment, the biocomplex comprises the TCE bound to a T-cell and a cancer cell, and the cancer cell is a gastrointestinal (GI)-cancer cell.

In a further aspect, the application provides methods for evaluating the efficacy of a T cell engager (TCE). In one embodiment, the application provides the steps of attaching a plurality of cells expressing at least a cancer marker to a cell culture plate, introducing a plurality of T-cells into the cell culture medium and introducing a TCE into the cell culture medium. In one embodiment, the TCE has a first binding specificity towards the cancer marker and a second binding specificity towards the T-cells, therefore the TCE is configured to bind the T-cells and the cells expressing at least a cancer marker.

The method further comprises measuring the rate of the T-cells attaching to, or detaching from, the cells expressing at least a cancer marker, or measuring the rate of the T-cells attaching to, or detaching from, the cell-culture plate.

In another embodiment, the application provides the steps of attaching a plurality of cells expressing at least a cancer marker to a first cell culture plate and culturing the cells in a first cell culture medium for a cell culturing period, introducing a plurality of T-cells into the first cell culture medium and introducing a TCE into the first cell culture medium. In one embodiment, the TCE has a first binding specificity towards the cancer marker and a second binding specificity towards the T-cells, therefore the TCE is configured to bind the T-cells and the cells expressing at least a cancer marker.

The method further comprises the step of co-culturing the T-cells bond through the TCE to the cells expressing at least a cancer marker attached to the first cell-culture plate. At this state, T-cells may be attached to the first cell-culture plate through the plate-bound cells expressing at least a cancer marker.

In one embodiment, the method further comprises the steps of collecting activated T-cells detached from the first cell-culture plate (therefore suspended in the first cell culture medium supernatant) and growing the activated T-cells in a second cell culture plate comprising a second cell culture medium for an expansion culturing period to provide expanded activated T-cells, pre-labeling the T-cells with a fluorescent dye, and counting the number of expanded activated T-cells to provide a growth rate of T-cells.

In one embodiment, the method further comprises the steps of growing the activated T-cells in a third cell culture plate comprising the cells expressing at least a cancer marker for a co-growing period; and measuring the cytotoxicity of the activated T-cells by counting the cell death of the cells expressing at least a cancer marker.

In one embodiment, the TCE is a bispecific antibody. In one embodiment, the cells expressing a cancer marker are cancer cells.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows schematics of assays measuring attach/detachment rate of T cells to cancer cells driven by TCE. (A) T cells are added to adherent cancer cells, medium in the well is harvested for cell count at different timepoints. Number of unbound T cells in the medium decreases with time. This assay measures attachment rate of the T cells. (B) After attachment of T cells to the cancer cells, unbound T cells are washed away, leaving behind the bound cells. Fresh medium with TCE is added back and bound T cells slowly detaches. Medium in the well is harvested for cell count at different timepoints. Number of unbound T cells in the medium increases with time. This assay measures detachment rate of the T cells.

FIG. 2 depicts association and dissociation curves of T cells binding to cancer cells. (A) Cell-binding curves at various ARB202 concentrations. Higher antibody concentration results in faster association and slower dissociation. (B) Half-life is calculated from the curves and the same trend is observed. (C) Cell-binding curves of ARB203 and a D124A point mutant of lower affinity. The D124A mutant has similar association rate but faster dissociation. (D) Half-life is calculated from the curves and the D124A mutant dissociates twice as fast as ARB203;

FIG. 3 depicts affinity, cell binding kinetics and functionality of TCE variants. (A) ELISA affinity of anti-CD3 variants and linker variants of ARB202 to CDH17 and CD3 antigens. (B) Schematic diagram of linker variants. (C) Cell-binding curves of anti-CD3 variants. Faster association and slower dissociation are observed with higher CD3 affinity. (D) Half-life is calculated from the curves and the same trend is observed. (E) Cytotoxicity and IFN-γ production of the variants. Higher CD3 affinity results in strong cytotoxicity and T cell activation while linker has no impact on functionality.

FIG. 4 shows repeat killing assay. (A) T cells are cultured with cancer cells in the presence of TCE for 3 days for killing, then harvested and cultured without cancer cells for 5 more days. These activated T cells are then subjected to a second round killing without TCE and calcein-AM assay is used to measure cytotoxicity. (B) Second round killing efficacy correlates with TCE concentration on first round; and

FIG. 5 shows T cell proliferation post-killing. (A) T cells are labelled with cell-tracking dye (e.g. CFSE) and cultured with cancer cells for 2 days for killing, then harvested and cultured without cancer cells for 5 more days. Viability and FITC fluorescence of the T cells are then analyzed with flow cytometry. (B) Significantly higher T cell count and lower FITC MFI is observed in ARB202 treatment groups, suggesting T cell proliferation after priming with cancer cells. (C) Dose-dependent increase in CFSE-low (divided) and Ki67+ T cells is observed in ARB203 post-killing proliferation.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present application is generally drawn, inter alia, to compositions, methods, apparatus, systems, devices, and/or computer program products related to the characterization of a plurality of T cells.

In a T cell therapy, a plurality of cytotoxic T cells may be originally derived from peripheral blood mononuclear cells (PBMC) that are subjected to the regulation by T cell receptor complex proteins, as well as co-stimulation signaling proteins, via either agonist receptors or antagonist receptors on their surface. As TCE antibodies shows promise in immunotherapy for treating cancer, many challenges remain. For example, a TCE antibody may bind to normal tissues expressing low levels of tumor associated antigen (TAA), leading to toxicities, i.e., its potential on-target off-tumor toxicity. Another well-known issue is associated with the control of T cell activation. Over-activation of T cells may lead to systemic inflammation, such as cytokine release syndrome that could be potentially life-threatening. In general, treating solid tumors is challenging due to the tumor heterogenicity and tumor microenvironment. Each designed TCE needs to be validated through multiple in vitro, ex vivo, and in vivo experimentations to show that each TCE antibody can penetrate matrix, contact tumor cells and bring T cell toxicity to tumor cells.

Besides the aforementioned challenges, studies on TCE have mainly been focused on short-term killing by T cell attachment, whether the T cells can detach from target cells and clonally activate is unknown. If a T cell stays attached to a target cell during killing, it is likely to undergo apoptosis along with the target; if the T cell detaches, it can engage another target and may expand clonally. The formation of a flexible immune synapse that allows detachment is therefore critical to a TCE's short-term and long-term efficacy. To assess whether a TCE candidate can form a detachable immune synapse, the present application discloses an assay platform and the composition of a quantifiable method for biopharmaceutical companies to measure binding kinetics of T cells to target cells. Specifically, the disclosed method measures the long-term effects of TCE stimulation. Of multiple assay parameters, the measurement of disengagement of the effector may allow the clonal expansion of memory T cells against the tumor cells. Evidence of T cell memory development reveals a novel mechanism of action (MoA), which may have a practical implication on TCE therapeutics. In this context, development of T cell memory against tumor antigens not only improves immunosurveillance but also reduces the risk of recurrence after treatment.

The terms “a”, “an” and “the” as used herein are defined to mean “one or more” and include the plural unless the context is inappropriate.

The terms “peptide”, and “protein”, as used herein, are interchangeable and are defined to mean a biomolecule composed of amino acids linked by a peptide bond.

The term “antigen” refers to an entity or fragment thereof which can induce an immune response in an organism, particularly an animal, more particularly a mammal including a human. The term includes immunogens and regions thereof responsible for antigenicity or antigenic determinants.

The term “antibody” is used in the broadest sense and specifically covers single monoclonal antibodies and/or recombinant antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody may be monoclonal, polyclonal, chimeric, single chain, multi-specific or multi-effective, human and humanized antibodies, as well as active fragments thereof. Examples of active fragments of molecules that bind to known antigens include Fab, F(ab′)₂, scFv and Fv fragments, including the products of a Fab immunoglobulin expression library and epitope-binding fragments of any of the antibodies and fragments mentioned above.

The term “Fv” refers to the minimum antibody fragment that contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

In some embodiments, antibody may include immunoglobulin molecules and immunologically active portions of immunoglobulin molecules, i.e. molecules that contain a binding site and that immunospecifically bind an antigen. A typical antibody refers to heterotetrameric protein comprising typically of two heavy (H) chains and two light (L) chains. Each heavy chain is comprised of a heavy chain variable domain (abbreviated as VH) and a heavy chain constant domain. Each light chain is comprised of a light chain variable domain (abbreviated as VL) and a light chain constant domain. The light chains of antibodies (immunoglobulins) from any vertebrate species can be assigned to one of two clearly distinct types, called kappa and lambda, based on the amino acid sequences of their constant domains. The VH and VL regions can be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by the hybridoma method first described by Kohler & Milstein, Nature, 256:495 (1975), or may be made by recombinant DNA methods (see, e.g., U.S. Pat. No. 4,816,567). “Recombinant” means the antibodies are generated using recombinant nucleic acid techniques in exogeneous host cells.

Monoclonal antibodies can be produced using various methods, including without limitation, mouse hybridoma, phage display, recombinant DNA, molecular cloning of antibodies directly from primary B cells, and antibody discovery methods (see Siegel. Transfus. Clin. Biol. 2002; Tiller. New Biotechnol. 2011; Seeber et al. PLOS One. 2014). Monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 [1984]).

The term “multi-specific” antibody as used herein denotes an antibody that has at least two binding sites each having a binding affinity to an epitope of an antigen. The term “bi-specific, tri-specific, tetra-specific, penta-specific, or hexa-specific” antibody as used herein denotes an antibody that has two, three, four, five, or six antigen-binding sites. For example, the antibodies disclosed herein with five binding sites are penta-specific, with six binding sites are hexa-specific.

The terms “cancer marker” and “tumor-associated antigen (TAA)” as used herein are interchangeable and are defined to mean anything present in or produced by cancer cells or other cells of the body in response to cancer or certain benign (noncancerous) conditions that provides information about a cancer, such as how aggressive it is, what kind of treatment it may respond to, or whether it is responding to treatment.

The terms “immune synapse” and “immunological synapse” as used herein are interchangeable and are defined to mean the interface between an antigen-presenting cell or target cell and a lymphocyte such as a T cell, B cell, or natural killer cell.

The terms “T cell engager”, “T-cell engager”, and “TCE” as used herein are interchangeable and are defined to mean the therapeutics of T cell-engaging therapy. TCEs are antibodies engineered to redirect the immune system's T cells to recognize and kill cancer cells. They are designed to bind to a target antigen expressed on a cancer cell and to a trigger molecule on T cells, such as CD3. These therapeutic molecules then engage T cells that are present in tumors but not capable of recognizing cancer cells, redirecting their activity toward the tumor.

Bi-specific T-cell engagers (BiTEs) are a class of artificial bispecific monoclonal antibodies that are investigated for use as anti-cancer drugs. They direct a host's immune system, more specifically the T cells' cytotoxic activity, against cancer cells. BiTE is a registered trademark of Micromet AG (fully owned subsidiary of Amgen Inc). BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to a tumor cell via a tumor specific molecule. Like other bispecific antibodies, and unlike ordinary monoclonal antibodies, BiTEs form a link between T cells and tumor cells. This causes T cells to exert cytotoxic activity on tumor cells by producing proteins like IFNγ, perforin and granzymes, independently of the presence of MHC I or co-stimulatory molecules. These proteins enter tumor cells and initiate the cell's apoptosis.

The first BITE, blinatumomab (Amgen, BLINCYTO®), was approved by FDA for the treatment of patients with relapsed and/or refractory B cell-precursor acute lymphoblastic leukemia (B-ALL). To broaden the indications for BiTE therapy from hematopoietic malignancies to common solid cancers, new and more-effective TCE therapies and rational combinatorial approaches are under investigation, including bifunctional checkpoint-inhibitory T cell engagers (CiTEs), simultaneous multiple interaction T cell engagers (SMITEs), trispecific killer engagers (TrikEs) and BiTE-expressing chimeric antigen receptor (CAR) T cells (CART.BiTE cells). Elucidation of mechanisms of resistance and immune escape and new technologies used in drug development pave the way designed to integrate various immune functions into one molecule or a single cellular vector and thereby enhance efficacy without compromising safety.

The present disclosure may be understood more readily by reference to the following detailed description of specific embodiments and examples included herein. Although the present disclosure has been described with reference to specific details of certain embodiments thereof, it is not intended that such details should be regarded as limitations upon the scope of the disclosure.

### EXAMPLES

### Example 1. Bispecific TCE: ARB202, ARB203, and Variants

Representing TCE, BiTE molecules are bispecific antibodies designed to form a bridge between cancer cells and cytotoxic T cells. A typical bispecific BiTE antibody has one domain for binding CD3, which is a protein complex and T cell co-receptor involved in activating both the cytotoxic T cell (CD8+ naive T cells) and T helper cells (CD4+ naive T cells), and another domain for binding a tumor-associated antigen (TAA). To demonstrate how the attachment-detachment assay works, two sets of bispecific antibodies were used. The first set includes ARB202, a CDH17/CD3 bispecific antibody (see WO2019222428A1, incorporated herein in its entirety) and its CD3 variants and linker variants (see Table 1). The second set includes ARB203, a Trop2/CD3 bispecific antibody (see WO2021113748A1, incorporated herein in its entirety) and its low-affinity mutant (with D124A).

For linker variants, the linker sequences were adapted from a published article (Chen, Zaro & Shen, 2013). For the generation of antibody variants with lower affinity, point mutations were made at the paratopes. The paratopes were deduced by proABC-2, an algorithm that predicts the amino acid residues of an antibody that are involved in antibody-antigen interactions (Ambrosetti et al., 2020).

Antigen binding of the variants was characterized by ELISA. A fixed amount of an antigen is coated on the wells. The antigen is then incubated with an antibody, at a range of mole ratios. EC50 is then calculated based on the signal given out by the bound antibody. To characterize the cytotoxicity of variants, cancer cells expressing the biomarker of interest were co-cultured with T cells, in the presence of an antibody at a range of different concentrations. The viability of cancer cells at certain end point was assessed by calcein-AM staining. Supernatant of the co-culture was also harvested, such that IFN-γ released by T cells during the co-culture could be quantified using ELISA.

### Example 2. Attachment/Detachment Kinetics

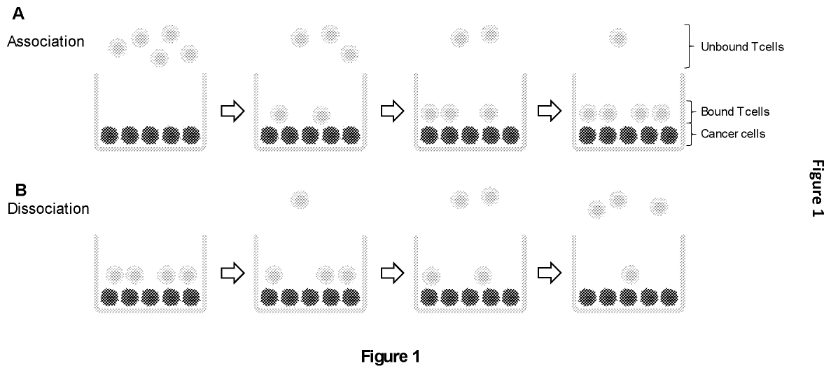

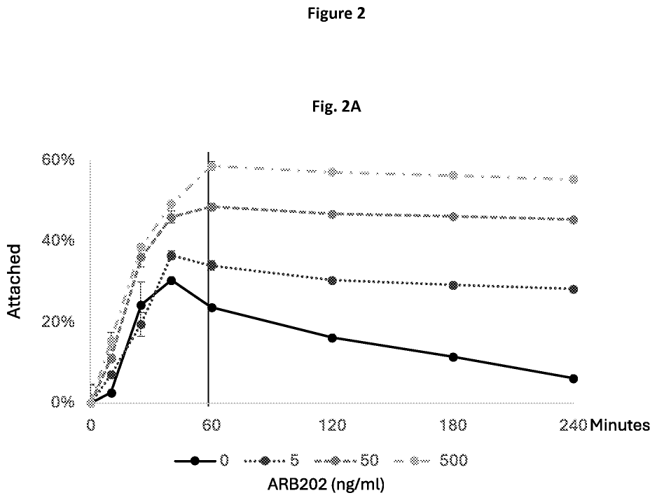

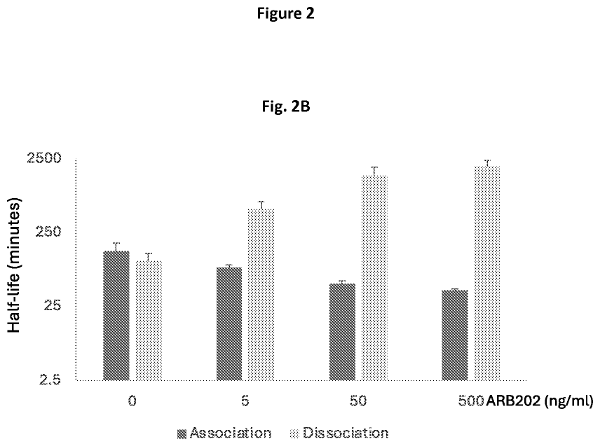

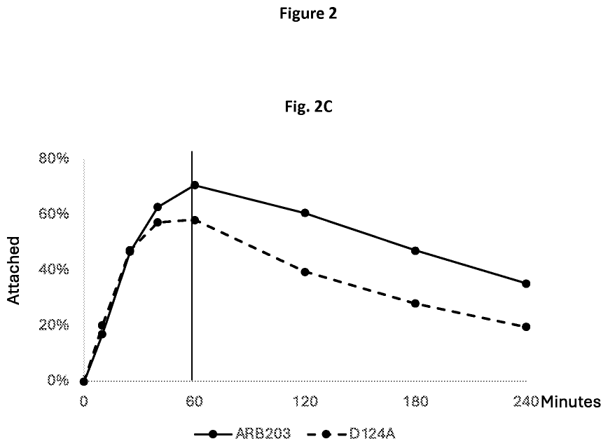

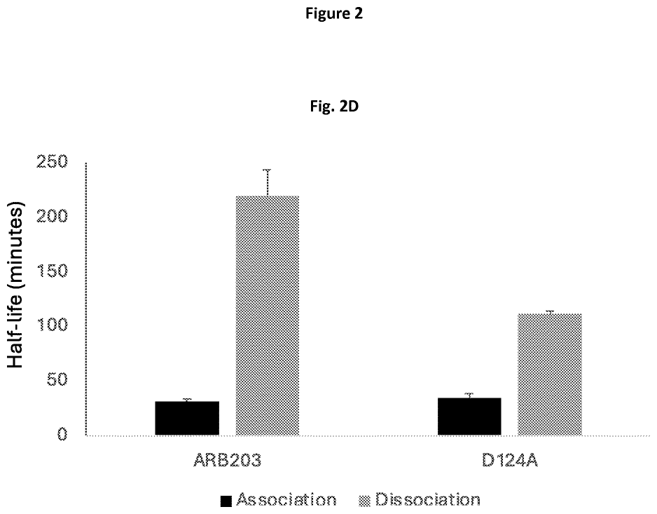

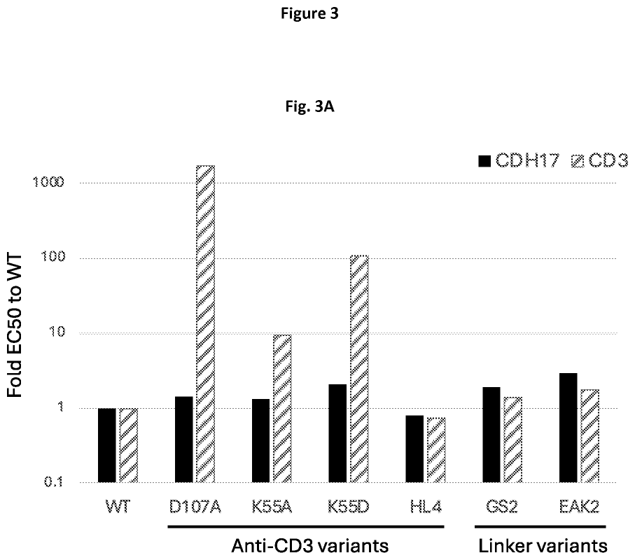

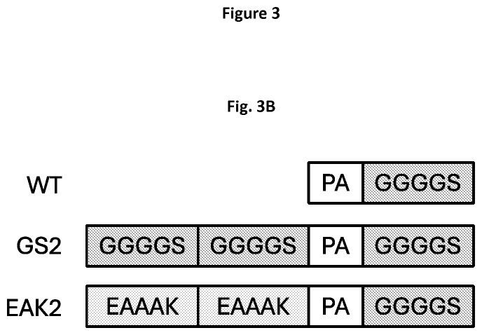

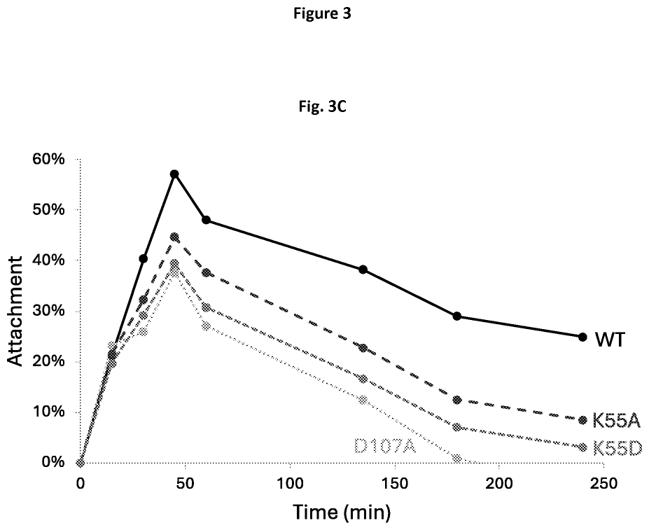

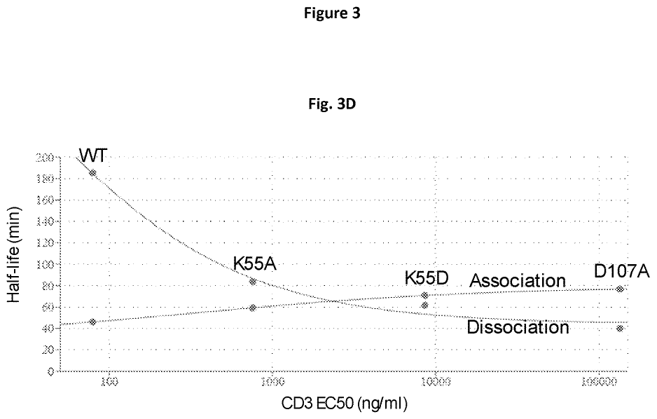

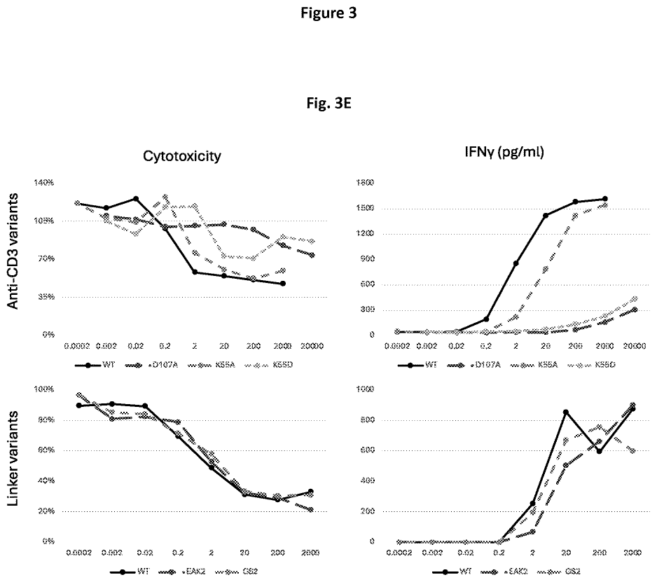

Cancer cells are seeded in a well and fluorescently labelled T cells are added along with TCE. T cells will bind to cancer cells and therefore reducing the unbound, free-floating T cells in the medium. At desired timepoints, medium is harvested, and cell count is performed with flow cytometry to calculate the fraction of T cells attached (FIG. 1A). For detachment kinetics, T cells are first allowed to attach for one hour, then unbound cells are removed and fresh medium with TCE is replaced. Bound cells will slowly detach into the new medium and at timepoints medium is harvested for cell count (FIG. 1B). Half-life of attach/detachment can be calculated from the curves generated. The assay demonstrated dose-dependent association and dissociation of T cells to DLD1 cells by ARB202, a CDH17/CD3 bispecific antibody (FIGS. 2A and 2B). A Trop2/CD3 bispecific antibody, ARB203, and its lower Trop2-affinity point mutant D124A, also demonstrated 2-fold difference in dissociation but no difference in association rate (FIGS. 2C and 2D). On the other hand, for anti-CD3 variants of ARB202 with decreasing affinities to CD3 (FIG. 3A), decreasing association and increasing dissociation of T cells to CDH17+ cancer cells are observed (FIGS. 3B and 3C). This is in concordance to the decreasing cytotoxicity and IFN-γ production as observed in the anti-CD3 variants (FIG. 3E). Besides anti-CD3 variants, linker variants of ARB202 are also generated (FIG. 3B). These linker variants associate T cells with and dissociate T cells from CDH17+ cancer cell lines at a rate similar to that of the wild type ARB202 (FIG. 3A). This matches to the fact that these linker variants have similar cytotoxicity to the wild type ARB202 upon co-culture with CDH17+ cancer cell lines (FIG. 3E). Table 1 summarizes the characterization of ARB202 with its CD3-variants as well as linker variants for comparison, including the CD3/CDH17-binding EC50, association half-lives, dissociation half-lives, cytotoxicity EC50, and IFN-γ EC50, as measured by fold differences.

### Example 3. Repeated Cytotoxicity

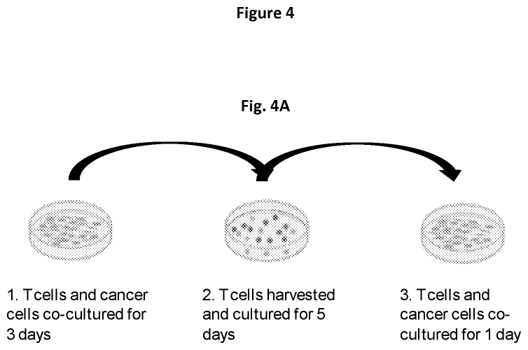

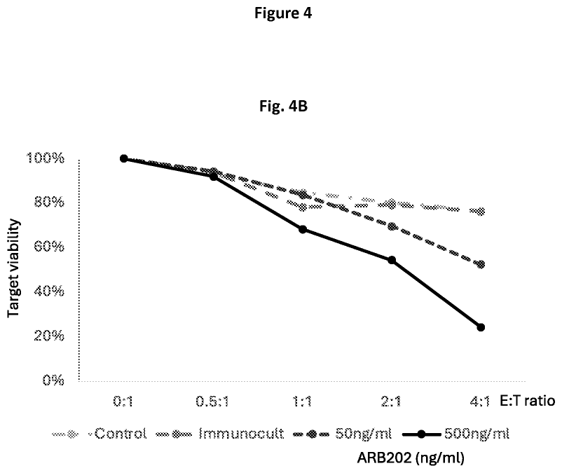

T cells and cancer cells are co-cultured with TCE for 24 hours for killing to take place, afterwards T cells in the medium is harvested and cultured in RPMI medium with IL-2 for up to one week. A second round of co-culture of primed T cells and cancer cells without TCE is done at a range of E:T for 24 hours (FIG. 4A). Amount of live cancer cells is determined by calcein-AM staining. T cells activated with higher concentrations of ARB202 in the first round demonstrated stronger cytotoxicity in the second round in the absence of ARB202 (FIG. 4B).

### Example 4. T Cell Proliferation after Killing

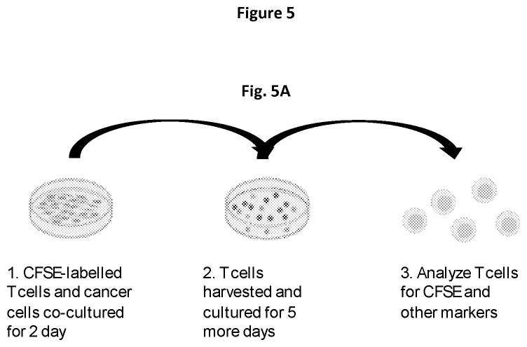

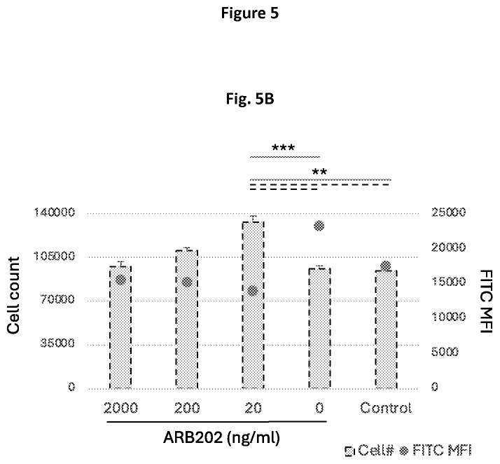

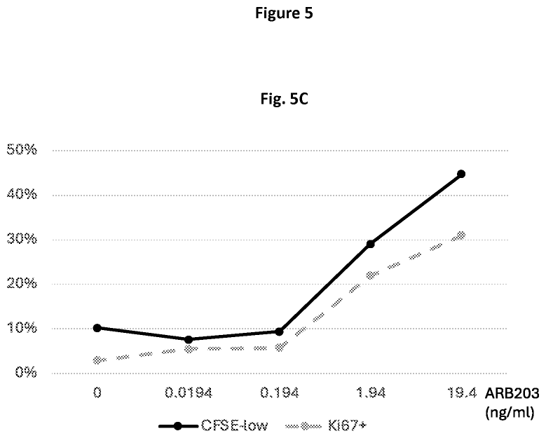

T cells are stained with CFSE and co-cultured with cancer cells with TCE for 24 hours for killing to take place, afterwards T cells in the medium is harvested and cultured in RPMI medium with IL-2 for up to one week. T cells are harvested, and flow cytometry is used to analysis viability and CFSE signal in the remaining live cells (FIG. 5A). Dose-dependent increase of live T cell count and decrease in mean fluorescence suggested ARB202-activation led to T cell expansion (FIG. 5B); while dose-dependent increase in CFSE-low and Ki67+ T cells suggested the ability of ARB203 in inducing T cell proliferation as well as activation (FIG. 5C).

### Example 5. Materials, Methods, and Protocols

### Attachment-Detachment Assay

1. In a 48-well plate, seed n wells with 30000-120000 cancer cells expressing the biomarker of interest, where the seeding density is at least 30000 per 1 cm², and n=the number of timepoints involved in the assessment of attachment kinetics.
2. Stain T cells with a fluorescent dye. Add the stained T cells with 1-2000 ng/mL TCE to the wells, where the number of T cells should not exceed ⅔ the number of cancer cells seeded. Incubate the cells at 37° C., 5% CO2.

### [for Assessment of Attachment Kinetics]

3. At desired time points within the first hour after the T cells are added, harvest the entire supernatant from one of the wells each time. Wash the well with 100-400 ul PBS. Harvest the PBS and combine it with the supernatant.

### [for Assessment of Detachment Kinetics]

4. Fresh medium containing the same concentration of TCE is added back to the well for assessing attachment kinetics at the last time point. Incubate the cells at 37° C., 5% CO2.
5. At desired time points within the subsequent 3 hours, harvest the entire supernatant. Wash the well with 100-400 ul PBS. Harvest the PBS and combine it with the supernatant. Fresh medium containing the same concentration of TCE is added back to the same well.
6. Quantify the number of T cells in each sample by performing a round of flow cytometry to count the number of cells with fluorescent signals.

### Repeat Killing Assay

1. In a 24-well plate, seed each well with 60000-240000 cancer cells expressing the biomarker of interest.
2. Add PBMC at an effector-to-target ratio of 4:1, along with 1-2000 ng/ml TCE to the cancer cells. Incubate at 37° C., 5% CO₂ for up to 48 hours.
3. Harvest the PBMC. Maintain the PBMC at a cell density of 5e5-3e6 cells/mL, in RPMI medium with 400U/mL IL-2. Incubate at 37° C., 5% CO₂ for up to one week.
4. In a 96-well plate, seed each well with 10000-40000 cancer cells, the same cancer cell used during the first round of co-culture.
5. Add the primed PBMC at a range of effector-to-target ratios, up to 4:1. Incubate at 37° C., 5% CO2 for up to 24 hours.
6. Assess the target cell viability.

### Proliferation Assay

1. In a 24-well plate, seed each well with 60000-240000 cancer cells expressing the biomarker of interest.
2. Stain PBMC with a fluorescent cell tracing reagent. Add the stained PBMC at an effector-to-target ratio of 4:1, along with 1-2000 ng/ml TCE to the cancer cells. Incubate at 37° C., 5% CO₂ for up to 48 hours.
3. Harvest the PBMC. Maintain the PBMC at a cell density of 5e5-3e6 cells/mL, in RPMI medium with 400U/mL IL-2. Incubate at 37° C., 5% CO₂ for up to one week.
Assess cell proliferation by performing a round of flow cytometry to quantify the number of live PBMC as well as the average fluorescent signals carried by the live PBMC.

The system of TCE assays may be used to screen, identify, and characterize TCEs as candidate therapeutics. Additionally, the assaying systems disclosed herein may be used to screen, identify, and characterize other types of engagers by using other types of immune cells, including but not limited to, natural killer cells (NK), natural killer T cells (NKT), dendritic cells (DC), and B lymphocytes (B).

### REFERENCES

1. Ambrosetti, F., Olsen, T. H., Olimpieri, P. P., Jiménez-García, B., Milanetti, E., Marcatilli, P., & Bonvin, A. M. J. J. (2020). proABC-2: PRediction of AntiBody contacts v2 and its application to information-driven docking. Bioinformatics (Oxford, England), 36 (20), 5107-5108. https://doi.org/10.1093/bioinformatics/btaa644
2. Chen, X., Zaro, J. L., & Shen, W. C. (2013). Fusion protein linkers: property, design and functionality. Advanced drug delivery reviews, 65 (10), 1357-1369. https://doi.org/10.1016/j.addr.2012.09.039

### TABLES

TABLE 1
summarizes the characterization of ARB202 with its CD3-variants as
well as linker variants for comparison, including the CD3/CDH17-
binding EC50, association half-lives, dissociation half-lives, cytotoxicity
EC50, and IFN-γ EC50, as measured by fold differences.

| | WT | D107A | K55A | K55D | HL4 | EAK2 | GS2 |
|---|---|---|---|---|---|---|---|
| CDH17 ELISA EC50 | 1 | 1.45 | 1.33 | 2.08 | 0.81 | 1.90 | 2.92 |
| CD3 ELISA EC50 | 1 | 1695.85 | 9.54 | 108.36 | 0.74 | 1.38 | 1.79 |
| Association t½ | 1 | 1.66 | 1.29 | 1.54 | | 1.12 | 1.02 |
| Dissociation t½ | 1 | 0.22 | 0.45 | 0.33 | | 1.06 | 1.21 |
| Cytotoxicity EC50 | 1 | 834.74 | 4.87 | 30.00 | | 1.61 | 3.02 |
| IFNγ EC50 | 1 | 2376.60 | 11.01 | 29739.90 | | 1.27 | 4.01 |

What is claimed is:

1. An assaying system for evaluating the efficacy and safety of a T cell engager (TCE), comprising:
a cell culture plate comprising at least one chamber for seeding a plurality of cells expressing at least a cancer marker in a cell culture medium at a density of at least 30,000 cells per cm² per chamber,
wherein the cell culture medium further comprises a plurality of T-cells at a cell number not more than ⅔ that of the cells expressing at least a cancer marker and a TCE at a concentration of at least 1 ng per ml of the cell culture medium,
wherein the TCE has a first binding specificity to the cancer marker and a second binding specific to the T-cells.

2. An assaying system for evaluating the efficacy of a T cell engager (TCE), comprising:
a first cell culture plate comprising at least one chamber for seeding a plurality of cells expressing at least a cancer marker in a first cell culture medium at a density of at least 30,000 cells per cm² per chamber,
wherein the first cell culture medium further comprises a plurality of T-cells at a cell number 4 times that of the cells expressing at least a cancer marker and a TCE at a concentration of at least 1 ng per ml of the first cell culture medium,
wherein the TCE has a first binding specificity to the cancer marker and a second binding specific to the T-cells, and
wherein the cells expressing at least a cancer marker, the T-cells, and the TCE are co-cultured for a co-culturing period to provide activated T-cells.

3. The assaying system of claim 2, wherein the TCE is configured to bind the T-cells and the cells expressing at least a cancer marker to provide a biocomplex, therefore activating T-cells, killing the cells expressing at least a cancer marker, and causing the cells expressing at least a cancer marker to detach from the first cell culture plate.

4. The assaying system of claim 2, further comprising a second cell culture plate for measuring growth rate of the activated T-cells,
wherein the second cell culture plate comprising a second cell culture medium,
wherein the activated T-cells are removed from the first cell culture medium and cultured in the second cell culture medium for an expansion culturing period to provide expanded activated T-cells,
wherein the growth rate is measured by pre-labeling the T-cells with a fluorescent dye and counting the number of expanded activated T-cells.

5. The assaying system of claim 2, further comprising a third cell culture plate for measuring cytotoxicity of the activated T-cells,
wherein the third cell culture plate comprising the cells expressing at least a cancer marker in a third cell culture medium,
wherein the activated T-cells are removed from the second cell culture medium and co-grown in the third cell medium with the cells expressing at least a cancer marker for a co-growing period, and
wherein the cytotoxicity of the activated T-cells is measured by counting the cell death of the cells expressing at least a cancer marker.

6. The assaying system of claim 1 or 2, wherein the cells expressing a cancer marker comprise cancer cells.

7. The assaying system of claim 1 or 2, wherein the cancer marker comprises CDH17.

8. The assaying system of claim 6, wherein the cancer cells comprise gastrointestinal cancer cells, gastric cancer cells, colon cancer cells, esophagi cancer cells, pancreatic cancer cells, liver cancer cells, or a combination thereof.

9. The assaying system of claim 1 or 2, wherein the T-cells comprise T lymphocytes derived from peripheral blood mononuclear cells, cytotoxic T cells, helper T cells, or a combination thereof.

10. The system of claim 1 or 2, wherein the T-cells express CD3.

11. The assaying system of claim 1 or 2, wherein the TCE comprises a bi-specific antibody or a multi-specific antibody.

12. The assaying system of claim 11, wherein bispecific antibody has a binding specificity to CD3 and CDH17.

13. A biocomplex of claim 3, comprising the TCE bound to the T-cell and the cell expressing at least a cancer marker.

14. The biocomplex of claim 13, wherein the cell expressing at least a cancer marker is a cancer cell.

15. The biocomplex of claim 14, wherein the cancer cell is a gastrointestinal (GI)-cancer cell.

16. A method for evaluating the efficacy and safety of a T cell engager (TCE), comprising the steps of:
attaching a plurality of cells expressing at least a cancer marker to a cell culture plate;
introducing a plurality of T-cells into the cell culture medium;
introducing a TCE into the cell culture medium, wherein the TCE has a first binding specificity towards the cancer marker and a second binding specificity towards the T-cells, wherein the TCE is configured to bind the T-cells and the cells expressing at least a cancer marker; and
measuring rate of the T-cells attaching to, or detaching from, the cells expressing at least a cancer marker.

17. A method for evaluating the efficacy and safety of a T cell engager (TCE), comprising the steps of:
attaching a plurality of cells expressing at least a cancer marker to a first cell culture plate and culturing the cells in a first cell culture medium for a cell culturing period;
introducing a plurality of T-cells into the first cell culture medium;
introducing a TCE into the first cell culture medium, wherein the TCE has a first binding specificity towards the cancer marker and a second binding specificity towards the T-cells, wherein the TCE is configured to bind the T-cells and the cells expressing at least a cancer marker; and
co-culturing the T-cells bond through the TCE to the cells expressing at least a cancer marker attached on the first cell-culture plate.

18. The method of claim 17, further comprising collecting T-cells detached from the first cell-culture plate to provide activated T-cells.

19. The method of claim 18, further comprising the steps of:
growing the activated T-cells in a second cell culture plate comprising a second cell culture medium for an expansion culturing period to provide expanded activated T-cells,
pre-labeling the T-cells with a fluorescent dye, and
counting the number of expanded activated T-cells to provide a growth rate of T-cells.

20. The method of claim 18, further comprising the steps of:
growing the activated T-cells in a third cell culture plate comprising the cells expressing at least a cancer marker in a third cell culture medium for a co-growing period; and
measuring the cytotoxicity of the activated T-cells by counting the cell death of the cells expressing at least a cancer marker.

21. The methods of claims 16 and 17, wherein the TCE is a bispecific antibody having an affinity to CD3 and CDH17.

22. The methods of claims 16 and 17, wherein the cells expressing a cancer marker are cancer cells.
